(19) United States
(12) Patent Application Publication
(10) Pub. No.: US 2018/0169297 A1
(43) Pub. Date: Jun. 21, 2018

(54) INJECTABLE COMPOSITION OF FACTOR VII AND FILLERS

(71) Applicant: Laboratoire Français du Fractionnement et des Biotechnologies, Les Ulis (FR)

(72) Inventors: Abdessatar Chtourou, Elancourt (FR); Jean-Luc Plantier, Croix (FR); Philippe Mondon, Neuve Chapelle (FR)

(73) Assignee: Laboratoire Français du Fractionnement et des Biotechnologies, Les Ulis (FR)

(21) Appl. No.: 15/735,270

(22) PCT Filed: Jun. 10, 2016
(86) PCT No.: PCT/EP2016/063357

§ 371 Date: Dec. 11, 2017

(30) Foreign Application Priority Data
Jun. 12, 2015 (EP) 15305909.2

(51) Int. Cl. A61L 27/20; A61K 8/73; A61L 27/22; A61K 8/66; A61K 8/64; A61K 8/42; A61Q 19/08

(52) U.S. Cl. CPC A61L 27/20; A61K 8/735; A61L 27/227; A61K 8/66; C12Y 304/21021; A61K 2800/91; A61K 8/64; C12Y 203/02013; A61K 8/42; A61Q 19/08; A61L 2300/402; A61L 27/225

(57) ABSTRACT

The present invention concerns an injectable composition comprising FVII and a filler, and its use for preventing or treating body and skin defects, especially folds, wrinkles, skin depressions and scars, while diminishing, decreasing or avoiding skin reactions due to injection, specially redness, ecchymosis, bruising, bleeding, erythema, oedema, necrosis, ulceration, swelling and/or inflammation.

### FIELD OF THE INVENTION

The present invention is in the dermatological domain. The present invention relates to an injectable composition comprising a filler, preferably hyaluronic acid, and Factor VII, preferably activated Factor VII. The invention also concerns a kit comprising syringe(s) and a composition according to the invention, and the use of said composition or said kit in the prevention or treatment of body and skin defects, in particular skin reactions due to injection. A method for preventing or treating body and skin defects, and a method for diminishing, decreasing or avoiding skin reactions due to injection are also provided.

### BACKGROUND OF THE INVENTION

Fillers such as hyaluronic acid are known as resorbable or slowly resorbable filling products, i.e. its effect is reversible since it will be degraded and absorbed by the body. It gives the possibility of filling structural body depressions, such as fine wrinkles, on the periphery of the mouth for example, but also deeper wrinkles like nasolabial folds.

However administration by injection of fillers, such as of hyaluronic acid, may cause red spots or blisters at the infiltrated areas and possibly bruises, or even bleeds at the needle-puncture site. The red spots on average persist for a few hours and the bruises for a week.

More significant bruising occurs with surgical procedures such as liposuction, breast augmentations/lifts, face lifts and tummy tucks.

The drawbacks mentioned above therefore pose problems, in particular within the scope of aesthetical interventions. It is observed that such drawbacks represent a physiological, aesthetical and moral inconvenience for the subject who has received the injections. In particular, the management of secondary immediate reactions due to dermal or intradermal injection of fillers with vascular damages or vascular breaking wall inducing ecchymosis, bruising, leakage of blood components having immediate action on inflammation setting up, redness and oedema, are of particular interest. Such drawbacks also generates apprehension or even unsatisfaction, related to the occurrence of red patches. In particular with regards to the consequences of bruising/bleeding, physicians report that one of the most significant concerns for patients is downtime as when bruising occurs, patients prefer to stay home rather than return to work and social activities.

Therefore, there is a need for alleviating bruising/bleeding that occur during aesthetic procedures, especially when fillers are injected.

Patent application WO 2010/136594 proposes to systemically deliver a dermal filler, in particular hyaluronic acid, combined with an adrenergic receptor agonist, in particular brimonidine, for its vasoconstriction properties.

### SUMMARY OF THE INVENTION

In order to find a remedy to the aforementioned drawbacks with further beneficial effects, the Applicants developed an injectable composition comprising a filler based on a coagulation factor and meeting several goals. The first object is to provide a novel injectable composition, with which it is possible to improve the body appearance, notably the appearance of the surface of the skin by reducing the depressions, such as wrinkles, or further by increasing the volume of certain portions of the body such as the lips. The second object is to alleviate physiological, aesthetical and moral inconveniences, notably to promote maintaining of hemostasis in order to significantly limit bleedings, occurrence of red patches, bruises.

Indeed, the present invention is based on the injection of Factor VII, in particular activated Factor VII (FVIIa), together with a filler, with improved appearance results. Advantageously, a composition according to the invention has improved filling properties, in particular an improved quality of the filler and an extended persistence of the filler in the patient. Additionally, a composition according to the invention has improved tolerance properties, reducing the occurrence of skin reactions due to injection.

The Factor VII (or factor VII or FVII) is a coagulation protein having the benefit of being able to locally act, once activated (“Factor VIIa” or “FVIIa”), in the presence of a released tissue factor after lesion of tissues generating hemorrhages, even in the absence of a Factor VIII or IX.

In a first aspect, the present invention concerns an injectable composition comprising a filler, preferably hyaluronic acid, and Factor VII.

The present invention also provides an injectable composition comprising a filler, Factor VII, and fibrinogen.

The present invention also provides an injectable composition comprising a filler, Factor VII, optionally fibrinogen and factor XIII.

In one embodiment, the present invention provides an injectable composition according to the invention, wherein at least one protein selected from said Factor VII, said fibrinogen and said FXIII, is recombinant.

In a more particular embodiment, the present invention provides an injectable composition according to the invention, wherein all the proteins selected from said Factor VII, said fibrinogen and said FXIII, are recombinant.

In another embodiment, the present invention provides an injectable composition according to the invention, wherein at least one protein selected from said Factor VII, said fibrinogen and said FXIII, is plasmatic.

The present invention also provides an injectable composition comprising a filler, Factor VII, optionally fibrinogen, optionally factor XIII, and a source of calcium ions.

Advantageously, an injectable composition according to the present invention further comprises an anesthetic agent, preferably lidocaine.

In a further aspect, the present invention concerns a kit comprising at least one syringe, preferably several syringes, and containing an injectable composition according to the invention. In a particular embodiment the syringe(s) may be prefilled with the composition to inject.

In still a further aspect, the invention concerns the use of a composition or a kit according to the present invention, in preventing or treating skin defects, specially folds, wrinkles, skin depressions and scars, while diminishing, decreasing or avoiding skin reactions due to the injection, specially redness, ecchymosis, bruising, bleeding, erythema, oedema, necrosis, ulceration, swelling and/or inflammation.

The composition or the kit according to the invention, is thus provided for use in diminishing, decreasing or avoiding skin reactions due to injection of the filler in a subject, preferably redness, ecchymosis, bruising, bleeding, erythema, oedema, necrosis, ulceration, swelling and/or inflammation, by injection to a subject.

The invention also provides a method for diminishing, decreasing or avoiding skin reactions due to injection of a filler, preferably redness, ecchymosis, bruising, bleeding, erythema, oedema, necrosis, ulceration, swelling and/or inflammation by injecting the subject with Factor VII, simultaneously or sequentially to the injection of the filler.

In a preferred embodiment, the filler and Factor VII are injected in a single composition, as defined herein.

Alternatively, the filler and Factor VII may be injected separately, either simultaneously or sequentially. In a particular embodiment, Factor VII is injected after injection of the filler. In another particular embodiment, Factor VII is injected before injection of the filler.

The present invention thus also provides a kit comprising a container containing an injectable composition of a filler, and a container containing an injectable composition of Factor VII.

### LEGENDS TO THE FIGURES

FIG. 1 shows activity of FVIIa (2 μg/ml) in presence of various hyaluronic acid concentrations.

FIG. 2 shows activity of FVIIa (4 μg/ml) in presence of various hyaluronic acid concentrations.

FIG. 3 shows the total time to clot for compositions comprising FVIIa (4 μg/ml) and fibrinogen (20 mg/ml) in presence of various concentrations of hyaluronic acid.

FIG. 4 shows efficiency of compositions comprising FVIIa (4 μg/ml) and fibrinogen (20 mg/ml) in presence of various concentrations of hyaluronic acid.

### DESCRIPTION OF THE INVENTION

### Injectable Compositions

Injectable compositions comprising a filler and/or Factor VII are provided. In a preferred embodiment, the invention relates to the combination of a filler and Factor VII in a single injectable composition. Unless otherwise specified in the present description, the term “composition” as used herein relates to any injectable composition comprising a filler and/or Factor VII.

The compositions are administered to a subject by injection, preferably by dermal injection, in particular by intradermal injection. Intradermal injections are delivered into the dermis (more precisely in the superficial, middle or deep dermis), or the skin layer underneath the epidermis (which is the upper skin layer). Thus, the definition of intradermal in the context of the present invention excludes the transdermal or subcutaneous injections. Therefore, in the context of the instant invention the filler and Factor VII are delivered to the target area of the skin in a pharmaceutically acceptable carrier. As used herein, a pharmaceutically acceptable carrier is any pharmaceutically acceptable formulation that can be applied to the skin for dermal, in particular for intradermal delivery of a pharmaceutical or medicament. The combination of a pharmaceutically acceptable carrier and a compound of the invention is designated an injectable formulation of the invention.

Typically, the composition consists in a solution or a gel, preferably an aqueous solution or gel.

The claimed composition is composed of or contains effective amounts of Factor VII and fillers. As used herein, an “effective amount” means the minimum amount of the compound that is effective to obtain the desired effect in the context of the invention.

The compositions used in the invention can comprise any other pharmaceutically acceptable components such as carriers, excipients, preservatives.

### The Filler

A filler is generally defined as a biomaterial able to fill dermal tissues. The composition to be injected, comprising said filler in an aqueous medium and displaying filling properties, can also be defined as a “dermal filler”. Within the scope of the invention, a filler can include a mix of different fillers.

In this context, compounds that can be used as dermal filler are resorbable polymer or molecules such as hyaluronic acid, collagen, alginate, dextran, elastine, polyurethane gels, chitosan, gelatin, carrageenans, or more permanent product as polyacrylamid gels, polymethylmethacrylate (PMMA) particles, microspheres or microparticles made of lactic acid polymers, glycolic acid polymers, or lactic acid-glycolic acid co-polymers, silicones, acrylic acid polymers, and derivatives thereof, this list not being exhaustive.

The most preferred compounds are resorbable molecules such as hyaluronic acid, collagen, alginate, dextran, elastin or polyurethane gels.

Within the injectable composition, the concentration of said filler is advantageously comprised between 0.5 mg/ml and 50 mg/ml, in particular between 10 mg/ml and 30 mg/ml. For example, the claimed composition comprises a concentration of filler of 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, or 30 mg/ml.

Thus, in an alternative way of measuring the quantity of the filler, the filler represents advantageously 0.5 to 5 weight by weight percent (w/w %) of the composition, in particular 1 to 3 w/w % of the composition. Within the scope of the invention, 10 mg/ml of the composition corresponds to 1 weight by weight percent (w/w %) of the composition. For example, the filler represents 1%, 1.1%, 1.2%, 1.3%, 1.4%, 1.5%, 1.6%, 1.7%, 1.8%, 1.9%, 2%, 2.1%, 2.2% 2.3% 2.4%, 2.5%, 2.6%, 2.7%, 2.8%, 2.9%, 3% expressed in w/w % of the composition.

In a preferred embodiment of the invention, the filler is hyaluronic acid.

Hyaluronic acid (HA) is a naturally occurring polysaccharide composed of a disaccharide motif comprising D-glucuronic acid and N-acetyl-D-glucosamine linked by alternating β(1,4)- and β(1,3)-glycosidic bonds.

Hyaluronic acid or hyaluronate is a non-sulfated glycosaminoglycan (GAG) widely distributed throughout connective, epithelial, and neural tissues. It is one of the chief components of the extracellular matrix. It contributes significantly to cell proliferation and migration. It plays an important role in skin hydration and skin elasticity. The level of hyaluronic acid decreases with ageing both in quantity and quality, inducing skin drying and wrinkles.

Hyaluronic acid and the other GAGs are negatively charged heteropolysaccharide chains which have a capacity to absorb large amounts of water and form highly viscous solutions in water. Therefore, it is widely used as a pharmaceutical product. Moreover, since hyaluronic acid is present with identical chemical structure except for its molecular mass in most living organisms, this compound is considered to be very safe and no immunogenicity reaction has been observed. So far, few minor adverse events have been noticed.

Therefore and advantageously, the filler is hyaluronic acid or a pharmaceutically acceptable salt or derivative thereof, particularly the sodium or potassium salt. Hyaluronic acid can be used under different forms: salts thereof, derivatives thereof such as esters or amides, in a linear form or cross-linked. In particular, the molecular weight, typically comprised between 500 kDa and 5 000 kDa, and the degree of cross-linking depends on the application, especially on the depth of the wrinkles to be filled.

In a particular embodiment, the filler is modified hyaluronic acid, e.g branched or crosslinked hyaluronic acid. Crosslinking and/or other modifications of the hyaluronic acid molecule is advantageous to improve its duration in vivo. Furthermore, such modifications can modify the liquid retention capacity of the hyaluronic acid molecule. According to certain embodiments the hyaluronic acid is a crosslinked hyaluronic acid. According to specific embodiments the hyaluronic acid is a hyaluronic acid gel.

Unless otherwise provided, the term “hyaluronic acid” encompasses all variants and combinations of variants of hyaluronic acid, hyaluronate or hyaluronan, of various chain lengths and charge states, as well as with various chemical modifications, including crosslinking. That is, the term also encompasses the various hyaluronate salts of hyaluronic acid with various counter ions, such as sodium hyaluronate. Various modifications of the hyaluronic acid are also encompassed by the term, such as oxidation, e.g. oxidation of —CH₂OH groups to —CHO and/or —COOH; periodate oxidation of vicinal hydroxyl groups, optionally followed by reduction, e.g. reduction of —CHO to —CH₂OH or coupling with amines to form imines followed by reduction to secondary amines; sulphation; deamidation, optionally followed by deamination or amide formation with new acids; esterification; crosslinking; substitutions with various compounds, e.g. using a crosslinking agent or a carbodiimide assisted coupling; including coupling of different molecules, such as proteins, peptides and active drug components, to hyaluronic acid; and deacetylation. Other examples of modifications are isourea, hydrazide, bromocyan, monoepoxide and monosulfone couplings.

The hyaluronic acid can be obtained from various sources of animal and non-animal origin. Sources of non-animal origin include yeast and preferably bacteria. The molecular weight of a single hyaluronic acid molecule is typically in the range of 0.1-10 MDa, but other molecular weights are possible.

In one embodiment embodiment, the hyaluronic acid is crosslinked.

Crosslinked hyaluronic acid comprises crosslinks between the hyaluronic acid chains, which creates a continuous network of hyaluronic acid molecules which is held together by the covalent crosslinks, physical entangling of the hyaluronic acid chains and various interactions, such as electrostatic interactions, hydrogen bonding and van der Waals forces.

Crosslinking of the hyaluronic acid may be achieved by modification with a chemical crosslinking agent. The chemical crosslinking agent may for example be selected from the group consisting of divinyl sulfone, multiepoxides and diepoxides. According to embodiments the chemical crosslinking agent is selected from the group consisting of 1,4-butanediol diglycidyl ether (BDDE), 1,2-ethanediol diglycidyl ether (EDDE) and diepoxyoctane. According to a preferred embodiment, the chemical crosslinking agent is 1,4-butanediol diglycidyl ether (BDDE).

The crosslinked hyaluronic acid product is preferably biocompatible. This implies that no, or only very mild, immune response occurs in the treated subject. That is, no or only very mild undesirable local or systemic effects occur in the treated subject.

The crosslinked hyaluronic acid product according to the invention may be a gel, or a hydrogel. That is, it can be regarded as a water-insoluble, but substantially dilute crosslinked system of hyaluronic acid molecules when subjected to a liquid, typically an aqueous liquid. While native hyaluronic acid and certain crosslinked hyaluronic acid products absorb water until they are completely dissolved, crosslinked hyaluronic acid gels typically absorb a certain amount of water until they are saturated, i.e. they have a finite liquid retention capacity, or swelling degree.

The gel contains mostly liquid by weight and can e.g. contain 90-99.9% water, but it behaves like a solid due to a three-dimensional crosslinked hyaluronic acid network within the liquid. Due to its significant liquid content, the gel is structurally flexible and similar to natural tissue, which makes it very useful as a scaffold in tissue engineering and for tissue augmentation.

As mentioned, crosslinking of hyaluronic acid to form the crosslinked hyaluronic acid gel may for example be achieved by modification with a chemical crosslinking agent, for example BDDE (1,4-butandiol diglycidylether). The hyaluronic acid concentration and the extent of crosslinking affects the mechanical properties, e.g. the elastic modulus G′, and stability properties of the gel. Crosslinked hyaluronic acid gels are often characterized in terms of “degree of modification”. The degree of modification (mole %) describes the amount of crosslinking agent(s) that is bound to HA, i.e. molar amount of bound crosslinking agent(s) relative to the total molar amount of repeating HA disaccharide units. The degree of modification reflects to what degree the HA has been chemically modified by the crosslinking agent. Reaction conditions for crosslinking and suitable analytical techniques for determining the degree of modification are all well known to the person skilled in the art, who easily can adjust these and other relevant factors and thereby provide suitable conditions to obtain a degree of modification in the range of 0.1-2% and verify the resulting product characteristics with respect to the degree of modification. The degree of modification of hyaluronic acid gels generally range between 0.1 and 15 mole %. A BDDE (1,4-butandiol diglycidylether) crosslinked hyaluronic acid gel may for example be prepared according to the method described in Examples 1 and 2 of published international patent application WO 9704012.

Within the claimed injectable composition, the concentration of hyaluronic acid is advantageously comprised between 0.5 mg/ml and 50 mg/ml, in particular between 10 mg/ml and 30 mg/ml. For example, the claimed composition comprises a concentration of hyaluronic acid of 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, or 30 mg/ml.

Thus, in an alternative way of measuring the quantity of hyaluronic acid, hyaluronic acid represents advantageously 0.5 to 5 weight by weight percent (w/w %) of the composition, in particular 1 to 3 w/w % of the composition. Within the scope of the invention, 10 mg/ml of the composition corresponds to 1 weight by weight percent (w/w %) of the composition. For example, the filler represents 1%, 1.1%, 1.2%, 1.3%, 1.4%, 1.5%, 1.6%, 1.7%, 1.8%, 1.9%, 2%, 2.1%, 2.2% 2.3% 2.4%, 2.5%, 2.6%, 2.7%, 2.8%, 2.9%, 3% as expressed in w/w % of the composition.

In a preferred embodiment the hyaluronic acid of the composition is present in the form of a crosslinked hyaluronic acid gel crosslinked by a chemical crosslinking agent, wherein the concentration of said hyaluronic acid is in the range of 10 to 30 mg/ml and the degree of modification with said chemical crosslinking agent is in the range of 0.1 to 2 mole %.

Hyaluronic acid gels may also comprise a portion of hyaluronic acid which is not crosslinked, i.e not bound to the three-dimensional crosslinked hyaluronic acid network. However, it is preferred that at least 50% by weight, preferably at least 60% by weight, more preferably at least 70% by weight, and most preferably at least 80% by weight, of the hyaluronic acid in a gel composition form part of the crosslinked hyaluronic acid network.

### Factor VII

The second component of the composition is Factor VII. The term “Factor VII” (or factor VII” or “FVII”) includes polypeptides comprising the 1-406 sequence of human wild-type human Factor VII (as disclosed in U.S. Pat. No. 4,784,950), or FVII derived from another species (e.g. bovine, porcine, canine, murine). It further comprises the natural allelic variations of Factor VII that may exist, and any form or degree of glycosylation or other post-translational modification. The term “Factor VII” also includes variants of Factor VII which has the same or higher biological activity compared to the activity of the wild form, these particular variants including polypeptides differing from the wild type Factor VIIa by insertion, deletion or substitution one or more amino acids.

“Factor VII” includes the uncleaved FVII (zymogen) and activated Factor VII. Factor VII is used in the composition preferably in its activated form (“FVIIa” or “activated FVII” or “activated factor VII”).

The term “biological activity of Factor VII” includes the ability to generate thrombin, for example on the surface of activated platelets. The tissue factor revealed in the wound of the patient will lead to form thrombin through the activation of coagulation.

Factor VII is generally a human Factor VII. It can be obtained in different ways, for example from the non cryoprecipitable fraction from human plasma or by genetic engineering from cells or from transgenic animals. Preferably, Factor VII (preferably in the form of Factor VIIa) is produced in particular in the transgenic animal milk, the formulation of the invention to keep the Factor VII satisfactory biological activity after lyophilization. According to a preferred embodiment, the human Factor VII is produced in the milk of nonhuman transgenic mammals, genetically engineered to produce this protein. Preferably it is the milk of a transgenic rabbit or goat. The secretion Factor VII by the mammary glands, allowing its secretion into the milk of the transgenic mammal, involves the control of the expression of the Factor VII-tissue-dependent manner. Such control methods are well known in the art. The expression control is performed using the sequences allowing expression of the protein to a particular tissue of the animal. These include promoter sequences WAP, beta-casein, beta-lactoglobulin and signal peptide sequences. In particular, an extraction process of proteins of interest from milk of transgenic animals is described in the patent EP 0 264 166.

In particular, Factor VII used in the scope of the invention can be human Factor VIIa produced in the milk of transgenic rabbit and compositions thereof, as described in Chevreux et al, Glycobiology, 2013 December; 23(12): 1531-46.

Alternatively, Factor VII can be produced by genetic engineering from BHK baby hamster kidney cells.

For example, Factor VII can be Factor VIIa NovoSeven®, authorized on the European market since 1996 and authorized on the American market in 1999, produced by the Danish company NovoNordisk. Factor VIIa can also be a variant of NovoSeven, called NovoSeven RT®.

Advantageously, composition according to the invention comprises Factor VIIa in a concentration comprised between 0.01 μg and 100 μg per milliliter of the final composition, preferably between 0.1 and 10 μg/ml, preferably between 1 and 5 μg/ml (corresponding to an activity comprised between 3.4 and 16.7 UI/ml), and more preferably between 2 and 4 μg/ml (corresponding to an activity comprised between 6.8 and 13.6 UI/ml). For example, the concentration of Factor VIIa within the claimed composition is 1 μg/ml, 1.5 μg/ml, 2 μg/ml, 2.5 μg/ml, 3 μg/ml, 3.5 μg/ml, 4 μg/ml, 4.5 μg/ml, or 5 μg/ml.

The composition comprising Factor VII is preferably formulated as a stable composition of Factor VII, and more particularly a stable composition of activated Factor VII. The term “stable composition” herein means that the formation of aggregates (insoluble or soluble) is minimized, and/or that the chemical degradation is reduced, the pH is maintained and the conformation of the protein is not substantially changed during the production or preservation of the compositions of the invention, such that the biological activity and stability of the protein is retained.

In a particular embodiment the filler is in the form of a gel, and Factor VII as a lyophilized powder may then be incorporated into the gel.

Stable compositions of lyophilized FVII are particularly described in the published patent application WO2010149907 and can be advantageously used herein. Such compositions comprise excipients such as a hydrophilic amino acid or amino acid bearing a positively charged side chain, such as arginine, a hydrophobic amino acid, an alkali metal salt, an alkaline-earth metal salt, and/or a salt of a transition metal.

According to a first embodiment, the claimed composition only contains a filler, or a mixture thereof, and Factor VII, advantageously hyaluronic acid and activated Factor VIIa.

### Additional Components

According to an alternative embodiment, the claimed composition also contains one or more additional components.

For instance the one or more additional components can be selected from fibrinogen, Factor XIII (FXIII), calcium ions, and anesthetics.

In one aspect, the additional component is fibrinogen, which is preferably combined with Factor VII. Thus, in one aspect, it is provided a composition which comprises a filler, factor VII and fibrinogen. Fibrinogen, the main structural protein in the blood responsible for the formation of clots, exists as a dimer of three polypeptide chains; the Aa (66.5 kD), Bβ (52 kD) and γ (46.5 kD) are linked through 29 disulphide bonds. The addition of asparagine-linked carbohydrates to the Bβ and γ chains results in a molecule with a molecular weight of 340 kD. Fibrinogen is proteolytically cleaved at the amino terminus of the Aa and BB chains releasing fibrinopeptides A and B (FpA & FpB) and converted to fibrin monomer by thrombin.

The term “fibrinogen” includes any natural allelic variations of fibrinogen that may exist, and any form or degree of glycosylation or other post-translational modification. Fibrinogen is naturally subject to phosphorylation, sulfation, and glycosylation. The term “fibrinogen” also includes variants of fibrinogen which has the same or higher biological activity compared to the activity of the wild form, these particular variants including polypeptides differing from the wild type fibrinogen by insertion, deletion or substitution one or more amino acids.

Fibrinogen, preferably virally secured, may be prepared by any partial or complete plasma fractionation known in the prior art. This may be the method described in EP 0 305 243 or further the one developed by the Applicant in patent application FR 2 887 883 according to which a fibrinogen concentrate may be obtained. It is also possible to apply transgenic (recombinant) fibrinogen produced in a cell line or via transgenic animals, notably in their milk. This may be transgenic fibrinogen produced and purified according to the method described in patent applications WO00/17234 or WO2009/134130.

The injectable composition according to the invention preferably includes a fibrinogen content of less than 60 mg per milliliter of injectable composition, in particular from 0.1 mg to 60 mg per ml of injectable composition, and on a more preferable basis from 0.1 mg/ml to 40 mg/ml, from 0.2 mg/ml to 40 mg/ml, from 0.5 mg/ml to 30 mg/ml, from 1 mg/ml to 25 mg/ml. For example, the injectable composition according to the invention can include 1 mg/ml of fibrinogen, 2 mg/ml of fibrinogen, 3 mg/ml of fibrinogen, 4 mg/ml of fibrinogen, 5 mg/ml of fibrinogen, 6 mg/ml of fibrinogen, 7 mg/ml of fibrinogen, 8 mg/ml of fibrinogen, 9 mg/ml of fibrinogen, 10 mg/ml of fibrinogen, 11 mg/ml of fibrinogen, 12 mg/ml of fibrinogen, 13 mg/ml of fibrinogen, 14 mg/ml of fibrinogen, 15 mg/ml of fibrinogen, 16 mg/ml of fibrinogen, 17 mg/ml of fibrinogen, 18 mg/ml of fibrinogen, 19 mg/ml of fibrinogen, 20 mg/ml of fibrinogen, 21 mg/ml of fibrinogen, 22 mg/ml of fibrinogen, 23 mg/ml of fibrinogen, 24 mg/ml of fibrinogen, 25 mg/ml of fibrinogen, 26 mg/ml of fibrinogen, 27 mg/ml of fibrinogen, 28 mg/ml of fibrinogen, 29 mg/ml of fibrinogen, or 30 mg/ml of fibrinogen.

Thus, in an alternative way of measuring the quantity of the fibrinogen, the fibrinogen represents advantageously 0.01 to 6 weight by weight percent (w/w %) of the composition, and on a more preferable basis from 0.01 to 4 w/w %, from 0.02 to 4 w/w %, 0.05 to 3 w/w %, 0.1 to 2.5 w/w % of the composition. In the scope of the invention, 10 mg/ml of the composition corresponds to 1 weight by weight percent (w/w %) of the composition. For example, fibrinogen represents 0.1%, 0.2%, 0.3%, 0.4%, 0.5%, 0.6%, 0.7%, 0.8%, 0.9%, 1%, 1.1%, 1.2%, 1.3% 1.4% 1.5%, 1.6% 1.7%, 1.8%, 1.9%, 2%, 2.1%, 2.2%, 2.3%, 2.4%, 2.5%, 2.6%, 2.7%, 2.8%, 2.9% or 3% as expressed in w/w % of the composition. The Applicant has observed that the best results in terms of the desired effects mentioned here above can be obtained when the contents of the two coagulation factors fibrinogen and Factor VIIa, and in particular their ratios, are specifically selected.

When combined with Factor VII, the ratio of the concentration of fibrinogen over the concentration of Factor VIIa (the concentrations being expressed in weight by volume) may be advantageously from 60,000:1 to 1,000:1, on a more preferable basis from 20,000:1 to 1,000:1 and in particular from 10,000:1 to 1,000:1.

In another aspect, the additional component is Factor XIII (‘factor XIII” or “FXIII”), which is also preferably combined with Factor VII. Thus, in one aspect, it is provided a composition which comprises a filler, Factor VII, optionally fibrinogen and Factor XIII.

An exogenous provision of Factor XIII promotes cross-linking of the fibrin network and therefore its coagulating and healing power.

The term “Factor XIII” includes any natural allelic variations of FXIII that may exist, and any form or degree of glycosylation or other post-translational modification. The term “Factor XIII” also includes variants of Factor XIII which has the same or higher biological activity compared to the activity of the wild form, these particular variants including polypeptides differing from the wild type fibrinogen by insertion, deletion or substitution one or more amino acids.

The Factor XIII, preferably virally secure, may be isolated from plasma by any method developed in the prior art and it may advantageously form the accompanying protein of fibrinogen during fractionation of the plasma. In this case, it is preferred to apply the method described in patent application FR 05 06640. It is also possible to apply the recombinant Factor XIII produced in a mammalian or yeast cell line, or transgenic factor XIII, produced in the milk of transgenic animals. This component should however meet the same criteria of purity as mentioned above, notably relating to the presence of other plasma factors, if the Factor XIII originates from plasma. In a preferred embodiment, Factor XIII according to the invention is plasmatic Factor XIII, purified from plasma.

Preferably the injectable composition comprises Factor XIII when the fibrinogen is recombinant. As may be necessary, the Factor XIII is advantageously present in an amount of 1 IU per milliliter to 700 IU per ml of final solution of the injectable composition, preferably from 2 IU/ml to 600 IU/ml, more preferably from 2 IU/ml to 500 IU/ml, more preferably from 2 IU/ml to 400 IU/ml, more preferably from 2 IU/ml to 300 IU/ml, more preferably from 2 IU/ml to 200 IU/ml, more preferably from 2 IU/ml to 100 IU/ml, more preferably from 2 IU/ml to 10 IU/ml. For example, as may be necessary, the factor XIII is advantageously present in an amount of 2, 3, 4, 5, 6, 7, 8, 9, or 10 IU/ml of final solution of the injectable of the composition.

Factor VII, fibrinogen and Factor XIII can be produced by recombinant techniques or purified from plasma. Thus, in one embodiment of the invention, at least one protein selected from Factor VII, fibrinogen and Facto XIII is recombinant. In one particular embodiment, all the proteins selected from Factor VII, fibrinogen and Factor XIII are recombinant. Within the scope of the invention, recombinant means expressed from recombinant construct in cell culture, a transgenic cell or in vitro. Recombinant proteins expressed in non-human cells or a non-human animal or in a non-human in vitro expression system can be made completely free of other human proteins. Recombinant proteins can also be entirely free of pathogens that may be present in the plasma.

Advantageously, the claimed composition comprises a source of calcium ions, which is preferably combined with Factor VII. Preferably, the composition comprises from 1 μmole to 30 μmoles of calcium ions par ml of the composition. A source of calcium ions is advantageously used as a cofactor of Factor VII, to improve the functional activity of Factor VII.

The sources of calcium ions represent water-soluble components, which are compatible with pharmaceutical use thereof. Preferably, these components present in the novel composition according to the invention are inorganic salts, such as calcium chloride (CaCl₂) or calcium gluconate. As may be necessary, the injectable composition includes from 1 micromole (μmole) to 30 μmoles of the source of calcium ions per ml of injectable composition, on a preferable basis from 1 to 6 μmoles/ml of the calcium ion source, on a more preferable basis from 3 to 6 μmoles/ml of the calcium ion source. For example, the injectable composition according to the invention includes 1, 2, 3, 4, 5 or 6 μmoles/ml of the calcium ion source.

In one advantageous embodiment, an additional component is an anesthetic. Thus, in one aspect, the injectable compositions comprise an anesthetic, in particular a local anesthetic selected from the group consisting of amide and ester type local anesthetics or a combination thereof. A local anesthetic is a drug that causes reversible local anesthesia and a loss of nociception. When it is used on specific nerve pathways (nerve block), effects such as analgesia (loss of pain sensation) and paralysis (loss of muscle power) can be achieved. The local anesthetic may be added to the hyaluronic acid composition to reduce pain or discomfort experienced by the patient due to the injection procedure. The groups of amide (also commonly referred to as aminoamide) type local anesthetics and ester (also commonly referred to as aminoester) type local anesthetics are well defined and recognized in the art.

Amide and ester type local anesthetic molecules are built on a simple chemical plan, consisting of an aromatic part linked by an amide or ester bond to a basic side-chain. The only exception is benzocaine which has no basic group. All other anesthetics are weak bases, with pKa values mainly in the range 8-9, so that they are mainly but not completely, ionized at physiological pH. As a result of their similarity they may be expected to have similar chemical and physical effects on the hyaluronic acid composition.

According to certain embodiments the local anesthetic is selected from the group consisting of amide and ester type local anesthetics, for example bupivacaine, butanilicaine, carticaine, cinchocaine (dibucaine), clibucaine, ethyl parapiperidinoacetylaminobenzoate, etidocaine, lignocaine (lidocaine), mepivacaine, oxethazaine, prilocaine, ropivacaine, tolycaine, trimecaine, vadocaine, articaine, levobupivacaine, amylocaine, cocaine, propanocaine, clormecaine, cyclomethycaine, proxymetacaine, amethocaine (tetracaine), benzocaine, butacaine, butoxycaine, butyl aminobenzoate, chloroprocaine, dimethocaine (larocaine), oxybuprocaine, piperocaine, parethoxycaine, procaine (novocaine), propoxycaine, tricaine or a combination thereof.

According to certain embodiments the local anesthetic is selected from the group consisting amide type local anesthetics, for example bupivacaine, butanilicaine, carticaine, cinchocaine (dibucaine), clibucaine, ethyl parapiperidinoacetylaminobenzoate, etidocaine, lignocaine (lidocaine), mepivacaine, oxethazaine, prilocaine, ropivacaine, tolycaine, trimecaine, vadocaine, articaine, levobupivacaine or a combination thereof. According to some embodiments the local anesthetic is selected from the group consisting of bupivacaine, lidocaine, and ropivacaine, or a combination thereof. According to specific embodiments the local anesthetic is lidocaine. Lidocaine is a well-known substance, which has been used extensively as a local anesthetic in injectable formulations, such as hyaluronic acid compositions.

The concentration of the amide or ester local anesthetic may be selected by the skilled person within the therapeutically relevant concentration ranges of each specific local anesthetic or a combination thereof. In certain embodiments the concentration of said local anesthetic is in the range of 0.1 to 30 mg/ml. In some embodiments the concentration of said local anesthetic is in the range of 0.5 to 10 mg/ml.

Preferably, the selected anesthetic is lidocaine. When lidocaine is used as the local anesthetic, the lidocaine may preferably be present in a concentration in the range of 1 to 5 mg/ml, more preferably in the range of 2 to 4 mg/ml, such as in a concentration of about 3 mg/ml.

Advantageously, the proportion of Factor VII to the filler (preferably HA) is comprised between 1:1000 and 1:300 000, preferably between 1:1000 and 1:100 000, more preferably between 1:1000 and 1:10 000 (weight/weight (w/w)). For example, the proportion of Factor VII to the filler (preferably HA) is 1:1000, 1:2000, 1:3000, 1:4000, 1:5000, 1:6000, 1:7000, 1:8000, 1:9000, or 1:10 000 (w/w).

Typically, the injectable composition contains:
a filler, preferably HA, representing 1 to 25 mg/ml of the composition;
Factor VIIa, representing 0.1 to 10 μg/ml of the composition;
optionally fibrinogen, representing 1 to 25 mg/ml of the composition;
optionally Factor XIII, representing 2 IU/ml to 10 IU/ml;
optionally a source of calcium ions, representing 2 to 30 μmole/ml;
optionally an anesthetic agent, representing 0.01% to 3% by weight of the composition

The components of the composition are solubilized in a water miscible solvent, preferably water for injection.

In preferred embodiments, injectable compositions comprising 20 mg/ml of filler, preferably HA, and 1 or 10 μg/ml of Factor VIIa are provided.

In some embodiments, each component of the compositions has been submitted to sterilization, before being mixed with any other component. In these embodiments, each component has been independently subjected to heat and/or steam and/or irradiation treatment in order to be sterilized.

The sterilization of each component is advantageously performed to conserve the functional activity of each component in the final composition according to the invention.

In alternative embodiments, the final compositions according to the invention have been subjected to sterilization, i.e. the final compositions according to the invention have been subjected to heat and/or steam and/or irradiation treatment in order to sterilize the composition. The sterilization of the composition is advantageously performed to conserve the functional activity of the final composition.

In some embodiments the final composition or each component of the composition has been subjected to sterilization by autoclaving or similar sterilization by heat or steam. Sterilization, e.g. autoclaving, may be performed at a F₀-value≥4. The F₀ value of a saturated steam sterilisation process is the lethality expressed in terms of the equivalent time in minutes at a temperature of 121° C. delivered by the process to the product in its final container with reference to micro-organisms posessing a Z-value of 10.

### Kits

Another aspect of the invention is an article of manufacture that comprises a formulation of the invention in a suitable container with labelling and instructions for use. The container is advantageously a single dose syringe.

Preferably, instructions are packaged with the formulations of the invention, for example, a pamphlet or package label. The labelling instructions explain how to administer formulations of the invention, in an amount and for a period of time sufficient to treat the patient. Preferably, the label includes the dosage and administration instructions, the formulation's composition, the clinical pharmacology, drug resistance, pharmacokinetics, absorption, bioavailability, and contraindications. The injectable composition according to the invention can then be integrated into a kit comprising one or more syringes containing said composition.

In another particular embodiment the filler and Factor VII can be contained in separate syringes for sequential administration. In such embodiment, the filler, optionally in combination with an anesthetic agent, is contained in a first syringe, and Factor VII, optionally in combination with one or more component e.g. selected from fibrinogen, Factor XIII and calcium ions, is/are contained in a separate syringe. In a further particular embodiment, the anesthetic agent is contained in a separate syringe.

In a particular embodiment, the filler, preferably the cross-linked hyaluronic acid, or an aqueous composition thereof, may be provided in the form of a pre-filled syringe, i.e. a syringe that is pre-filled with a filler composition, preferably the cross-linked hyaluronic acid, and autoclaved.

The filler composition and the Factor VII composition can be used simultaneously, or separately in the context of the present invention.

When used simultaneously, the filler and Factor VII are preferably presented as a mixture contained in a single syringe. Alternatively they can be in the form of two separate compositions which are mixed extemporaneously, before injection.

When used separately, the filler and the Factor VII may be contained in at least two separate syringes which can be adapted for extemporaneous mixture, or which may be used sequentially.

For example, the filler is firstly administered to a subject in need thereof, and the Factor VII, is subsequently injected after at least one hour. For example, Factor VIIa, is administered 24 hours, 48 hours, 72 hours, 96 hours, 120 hours, 144 hours, 168 hours or 192 hours, after the injection of the filler, preferably hyaluronic acid.

In another embodiment, Factor VII is injected before the injection of the filler. For example, the Factor VII is firstly administered to a subject in need thereof, and the filler, is subsequently injected after at least one hour. For example, the filler, preferably hyaluronic acid, is administered 24 hours, 48 hours, 72 hours, 96 hours, 120 hours, 144 hours, 168 hours or 192 hours, after the injection of Factor VII, preferably Factor VIIa.

### Applications

The injectable compositions described herein are intended for use in preventing or treating body and skin defects, specially folds, wrinkles, skin depressions and scars. Such treatment is usually considered cosmetic, i.e. non-medical.

The claimed composition is meant to be administered to a subject or a patient, especially by facial injection (forehead, eyes, nasolabial fold, . . . ). As used herein, the term “subject” or “patient” are used equivalently and means any animal, preferably a mammal, more preferably, a human to whom will be or has been administered compounds or formulations of the invention. The term “mammals” used herein encompasses any mammal.

The use preferably comprises injecting the composition(s) into the cutis of a human subject, defined as the combination of the epidermal and the dermal outer layers of the skin. The use of the injectable composition(s) for improving the appearance of skin, filling wrinkles or contouring the face or body of a subject, may be essentially or totally non-medical, e.g. purely cosmetic.

When used separately, the composition of Factor VII is injected at substantially the same site as the composition of filler, or in its vicinity.

The injectable compositions comprising the filler are useful in, e.g., soft tissue augmentation, for example filling of wrinkles, by a filler injection, preferably a hyaluronic acid gel injection. The compositions have been found especially useful in a cosmetic treatment, referred to herein as skin revitalization, whereby small quantities of the filler composition are injected into the dermis at a number of injection sites distributed over an area of the skin to be treated, resulting in improved skin tone and skin elasticity. Skin revitalization is a simple procedure and health risks associated with the procedure are very low.

According to some aspects illustrated herein, there is provided the use of a composition as described above for cosmetic, non-medical, treatment of a subject by administration, preferably by dermal or intradermal injection, of the composition into the skin of the subject. A purpose of the cosmetic, non-medical, treatment may be for improving the appearance of the skin, filling wrinkles or contouring the face or body of a subject. The cosmetic, non-medical, use does not involve treatment of any form of disease or medical condition. Examples of improving the appearance of the skin include, but are not limited to, treatment of sun-damaged or aged skin, skin revitalization and skin whitening.

According to certain embodiments, there is provided the use of a composition as described above for improving the appearance of skin, filling wrinkles or contouring the face or body of a subject.

According to a preferred embodiment, there is provided the use of a filler composition as described herein, for skin revitalization.

The cosmetic compositions are administered by dermal or intradermal injection into the skin of a subject, preferably into the cutis.

Preferably the compositions are in the form of a gel.

Administration of gel structures may be performed in any suitable way, such as via injection from traditional hand-held syringes or any injection device for delivering liquid/viscous compositions, as described in patent EP2574357. Any syringe may be equipped with standard cannulae and needles of appropriate sizes or surgical insertion. The administration is performed where the soft tissue augmentation is desired, such as the chin, cheeks or elsewhere in the face or body.

For instance, the diameter of the injection needle ranges preferably from 7 to 34 gauge.

In an embodiment, the volume of the filler composition to be injected varies between 0.1 and 10 ml, typically between 0.5 and 4 ml. Preferably, said volume is presented as a single dose syringe. Said injection can be repeated, for example after 4 to 18 months.

There is provided a method for preventing or treating body and skin defects, specially folds, wrinkles, skin depressions and scars, by injecting in an subject in need thereof, comprising:
1) providing a filler composition, further comprising Factor VII
2) administering said composition into the skin of a subject

According to certain embodiments, the method comprises improving the appearance of skin.

According to a preferred embodiment, the method comprises skin revitalization.

According to certain embodiments, the method comprises filling wrinkles or contouring the face or body of a subject.

According to this method, the presence of Factor VII, acting alone or synergistically with the other components of the composition according to the invention, allows for diminishing, decreasing or avoiding skin reactions due to injection of the filler, preferably redness, ecchymosis, bruising, bleeding, erythema, oedema, necrosis, ulceration, swelling and/or inflammation.

The invention thus further provides a method for diminishing, decreasing or avoiding skin reactions due to injection of a filler, preferably redness, ecchymosis, bruising, bleeding, erythema, oedema, necrosis, ulceration, swelling and/or inflammation by injecting the subject with Factor VII, simultaneously or sequentially, e.g. subsequently, to the injection of the filler.

Other potential benefits of combining the filler and Factor VII, optionally with the other components according to the invention, for simultaneous or sequential use, are as follows:
By reducing the skin reactions, in particular inflammation, Factor VII, acting alone or synergistically with the other components of the composition according to the invention, allows the filler to persist longer, possibly due to its slower degradation: the more tissue reaction is severe, in particular the more inflammatory the filler is, and higher is the level of undesirable species (e.g. inflammatory species), thus degrading the filler faster.
When the composition further contains an anaesthetic, e.g. lidocaine, the efficiency of said anaesthetic is improved: Without being bound to a particular mechanism of action or theory, it is believed that the vasoconstrictive effect provided by the filler composition limits anaesthetic diffusion in a large area, thus making anaesthetic efficient in the strict injection site;
Without being bound to a particular mechanism of action or theory, it is believed that the vasoconstrictive effect of the composition of the invention, comprising the filler and FVII, also allows to concentrate Factor VII, optionally along with the other components according to the invention, at the site of injection, and that the local increase of Factor VII concentration leads to a reduction of blood loss, hence to a reduction of oedema and swelling.
After administration of the composition according to the invention, preferably by intradermal injection, a cell colonization of said composition can be observed from the injection site. It contributes to the overall efficiency of the dermal filler, in particular to the treatment of the skin defects.

More generally, Factor VII, combined with the filler, and optionally with the other components according to the invention, is intended to diminish, decrease or avoid all the undesirable skin reactions (immediate and/or secondary) due to injection. These include ecchymosis, bruising or bleeding but also possibly redness, erythema, oedema, necrosis, ulceration, swelling and inflammation.

In addition to the above, the following examples are provided to illustrate particular embodiments and not to limit the scope of the invention.

### EXAMPLES

### Example 1: An Example of Study Protocol

A test of compositions according to the invention comprising hyaluronic acid-based filler and Factor VII (TEST PRODUCTS) can be performed in order to evaluate the potential of said compositions to reduce the undesirable skin reactions following intradermal injection of filler in the rabbit. TEST PRODUCTS are defined as compositions according to the invention and containing:
1. Hyaluronic acid and activated Factor VIIa
2. Hyaluronic acid, activated Factor VIIa, and fibrinogen
3. Hyaluronic acid, activated Factor VIIa, and a source of calcium ions
4. Hyaluronic acid, activated Factor VIIa, and lidocaïne
5. Hyaluronic acid, activated Factor VIIa, fibrinogen, and a source of calcium ions
6. Hyaluronic acid, activated Factor VIIa, fibrinogen, and lidocaine
7. Hyaluronic acid, activated Factor VIIa, a source of calcium ions and lidocaine
8. Hyaluronic acid, activated Factor VIIa, fibrinogen, a source of calcium ions and lidocaine
9. Hyaluronic acid, activated Factor VIIa, fibrinogen, and Factor XIII
10. Hyaluronic acid, activated Factor VIIa, fibrinogen, Factor XIII and a source of calcium ions
11. Hyaluronic acid, activated Factor VIIa, fibrinogen, Factor XIII, a source of calcium ions and lidocaïne

Adult rabbits receive 0.2 mL of a composition according to the invention comprising hyaluronic acid and Factor VIIa, alone or with other components according to the invention (“TEST PRODUCTS”), NaCl 0.9% (negative control) and hyaluronic acid filler alone (positive control), by intradermal route. The results obtained with each TEST PRODUCT are compared with the positive control.

The sites are examined from Day 0 to Day 8 after injection for gross evidence of tissue reaction, such as erythema, oedema and necrosis and the observation of microscopic tissue response can be done on histological observations after sacrifice at Day 8.

The study is conducted according to the requirements of the ISO 10993 standard: Biological Evaluation of medical devices, Part 10: Test for irritation and delayed type hypersensitivity.

### MATERIALS AND METHODS

### Preparation of Control Crosslinked Hyaluronic Filler (Crosslinked HA)

The hyaluronic acid used in the present invention is prepared by conventional means as described above in order to obtain a crosslinked hyaluronic acid filler. Crosslinked hyaluronic acid are commercially available.

### Preparation of the Test Products: Composition Based on Hyaluronic Acid and Factor VIIa

Preferably the compositions are prepared by mixing a powder of Factor VIIa into a gel of crosslinked HA.

The following hyaluronic acid based gel compositions can be prepared in water:
1. Crosslinked HA is mixed with Factor VIIa to obtain a composition comprising:
   HA representing 1 to 25 mg/ml of the composition
   Factor VIIa representing 0.1 to 10 μg/ml of the composition
2. Crosslinked HA, activated Factor VIIa, and fibrinogen are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
3. Crosslinked HA, activated FVIIa, and a source of calcium ions are mixed to obtain a composition comprising:
   HA representing 1 to 25 mg/ml of the composition
   Factor VIIa representing 0.1 to 10 μg/ml of the composition
   a source of calcium ions, representing 2 to 30 μmole/ml
4. Crosslinked HA, Factor VIIa, and lidocaine are mixed to obtain a composition comprising:
   HA representing 1 to 25 mg/ml of the composition
   Factor VIIa representing 0.1 to 10 μg/ml of the composition
   lidocaine representing 0.01% to 3% by weight of the composition
5. Crosslinked HA, Factor VIIa, fibrinogen, and a source of calcium ions are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
   a source of calcium ions, representing 2 to 30 μmole/ml
6. Crosslinked HA, Factor VIIaFVII, fibrinogen, and lidocaine are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
   lidocaine representing 0.01% to 3% by weight of the composition
7. Crosslinked HA, Factor VIIa, a source of calcium ions and lidocaine are mixed to obtain a composition comprising:
   HA representing 1 to 25 mg/ml of the composition
   Factor VIIa representing 0.1 to 10 μg/ml of the composition
   a source of calcium ions, representing 2 to 30 μmole/ml
   lidocaine representing 0.01% to 3% by weight of the composition
8. Crosslinked HA, Factor VIIa, fibrinogen, a source of calcium ions and lidocaine are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
   a source of calcium ions, representing 2 to 30 μmole/ml
   lidocaine representing 0.01% to 3% by weight of the composition
9. Crosslinked HA, Factor VIIa, fibrinogen, and Factor XIII are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
   Factor XIII, representing 2 IU/ml to 10 IU/ml
10. Crosslinked HA, Factor VIIa, fibrinogen, Factor XIII and a source of calcium ions are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
   Factor XIII, representing 2 IU/ml to 10 IU/ml
   a source of calcium ions, representing 2 to 30 μmole/ml
11. Crosslinked HA, Factor VIIa, fibrinogen, Factor XIII, a source of calcium ions and lidocaïne are mixed to obtain a composition comprising:
   HA representing 1 to 25 mg/ml of the composition
   Factor VIIa representing 0.1 to 10 μg/ml of the composition
   fibrinogen, representing 0.1% to 2.5% by weight of the composition
   Factor XIII, representing 2 IU/ml to 10 IU/ml
   a source of calcium ions, representing 2 to 30 μmole/ml
   lidocaine representing 0.01% to 3% by weight of the composition

Preferably, the following compositions are tested:

### TEST PRODUCT 1a:

Crosslinked HA representing 20 mg/ml (2% weight by weight of the composition) and
Factor VIIa produced in the milk of transgenic rabbit representing 2 μg/ml of the composition

### TEST PRODUCT 1b:

Crosslinked HA representing 20 mg/ml (2% weight by weight of the composition) and
Factor VIIa produced in the milk of transgenic rabbit representing 5 μg/ml of the composition

### TEST PRODUCT 1c:

Crosslinked HA representing 20 mg/ml (2% weight by weight of the composition) and
Factor VIIa produced in the milk of transgenic rabbit representing 10 μg/ml of the composition

### TEST PRODUCTS 2a:

TEST PRODUCT 1a, TEST PRODUCT 1b or TEST PRODUCT 1c, and
plasma derived fibrinogen, representing 5 mg/ml of the composition

### TEST PRODUCTS 2b:

TEST PRODUCT 1a, TEST PRODUCT 1b or TEST PRODUCT 1c, and
plasma derived fibrinogen, representing 10 mg/ml of the composition

### TEST PRODUCTS 2c:

TEST PRODUCT 1a, TEST PRODUCT 1b or TEST PRODUCT 1c, and
plasma derived fibrinogen, representing 15 mg/ml of the composition

### TEST PRODUCTS 2d:

TEST PRODUCT 1a, TEST PRODUCT 1b or TEST PRODUCT 1c, and
plasma derived fibrinogen, representing 20 mg/ml of the composition

When needed in TEST PRODUCTS 3 to 8, the composition preferably tested contains 4 mM of a source of calcium ions.

### Investigation of In Vitro Filling Properties of the Test Products by Thromboelastometry (TEM)

TEM is performed with the ROTEM (rotational thromboelastometry) whole blood analyzer (Tem Innovations GmbH, Munich) and is an enhancement of thrombelastography, originally described by H. Hartert in 1948. TEM provides a systematic way to evaluate several parameters: the coagulation time (CT, sec) and the clot forming time (CFT, sec), two kinetic parameters characterizing the coagulation speed of a composition. For simplicity, the value corresponding to the sum of these two parameters (CT+CFT, sec) is evaluated, corresponding to the total time to clot. The system also provides a value that reflects the strength of the clot (maximum clotting firmness or MCF in millimeter). The overall efficiency of coagulation can be calculated as follows: MCF/(CF+CFT), mm/sec.

Briefly, the minimal plasma concentration required to clot is evaluated. The compositions according to the invention (TEST PRODUCTS) are prepared in a small cup ROTEM (500 μl). The experiment is then initiated by adding 0.5 pM of tissue factor, 4 mM of phospholipids, minimal human plasma concentration required to clot (2 to 8%) and 5 mM of CaCl₂.

### Results:

The total time of clot formation (CT+CFT) and the strength of the clot (MCF) are evaluated for each TEST PRODUCT. The overall efficiency of coagulation can be calculated as followed: MCF/(CF+CFT), mm/sec. This in vitro study can be advantageously used to observe the advantages of a composition according to the invention comprising Factor VIIa and a filler, in particular to evaluate the beneficial effect on the coagulation properties.

### Investigation of Immediate Adverse Events Reduction

The potential of irritation reduction by TEST PRODUCTS is evaluated in an animal study conducted according to the requirements of the ISO10993-10 requirements: Biological Evaluation of Medical Devices-Test for irritation and delayed type hypersensitivity.

### Study Protocol

At Day 0 (DO), two adult rabbits receive 0.2 mL of a composition comprising hyaluronic acid and Factor VIIa, as well as optional components according to the invention (“Test Products”), NaCl 0.9% (negative control) and positive control), injected using a 27G needle, by intradermal route. In these conditions, 4 sites are injected for each product.

Then, the injected sites are examined twice a day from Day 0 to Day 8 for gross evidence of tissue reaction such as erythema, oedema, ulceration and necrosis, attributing a score with the following criterions:

| Criterion | Control method | Scale |
|---|---|---|
| Formation of | Visual assessment | (0) none |
| oedema | | (1) slight |
| | | (2) moderate |
| | | (3) marked |
| | | (4) severe |
| Formation of | Visual assessment | (0) none |
| erythema, | | (1) slight |
| ulceration | | (2) moderate |
| and necrosis | | (3) marked |
| | | (4) severe |

At Day 8, the animals are euthanized and injection sites are collected and fixed for histological analysis. Microscopic analyses are performed to assess the following criterions:

| Criterion | | Control method | Scale |
|---|---|---|---|
| Type of cell/implant reaction, | | Histological | Score from 0 to 4 for |
| local tolerance: | | analysis | each criterion |
| ✓ | Fibrin | | (0) None |
| ✓ | Necrosis | | (1) slight |
| ✓ | Tissue degeneration | | (2) moderate |
| ✓ | Granulocyte | | (3) marked |
| ✓ | PMN eosinophils | | (4) severe |
| ✓ | Lymphocytes | | |
| ✓ | Plasmocytes | | |
| ✓ | Macrophages | | |
| ✓ | Giant cells | | |
| ✓ | Fibrocytes | | |
| ✓ | Neovessels | | |
| ✓ | Peri and intra-implant tissue | | |
| | reconstruction | | |
| ✓ | Degradation of the material | | |

### Exploitation of the Results

The occurrence of oedema, erythema, ulceration or necrosis in this study can be checked by visual assessment.

### 1. Irritation Primary Index (IPI)

IPI of test product is determined for each observation time in the following way:

$${IPI}_{Test} = \frac{Σ ( oedema score + erythema score )}{Observations number} - {IPI}_{negative control}$$

$$Negative control \text{:} physiological saline$$

$${IPI}_{negative control} quotes 0 for each observation time .$$

Value of irritation index during the experiment can thus be obtained.

### 2. Histological Analysis

Total scores of inflammation are determined from histological observations according to local tolerance-representative cells type and quantities.

### 3. Conclusion

This study can be advantageously used to observe the advantages of a composition according to the invention comprising Factor VII and a filler. In particular, this study can be used to determine the effectiveness of a composition according to the invention for diminishing or preventing adverse events due to intradermal injection.

### Example 2: Effect of Hyaluronic Acid (HA) on Biological Properties of Activated Factor VII (FVIIa)

First of all, the effect of HA on the FVIIa chromogenic activity was assessed in vitro. Recombinant FVIIa produced in the milk of transgenic rabbit (1 mg/ml) was diluted in_HEPES buffer (25 mM Hepes, 175 mM NaCl pH 7.4 in the presence of Tissue factor (Dade Innovin Siemens). Hyaluronic acid (25 mg/ml) (Sigma Aldrich, ref. 97616-50MG reconstituted in HEPES buffer) was added at the final concentrations of 20-15-10-5-2-0 mg/ml.

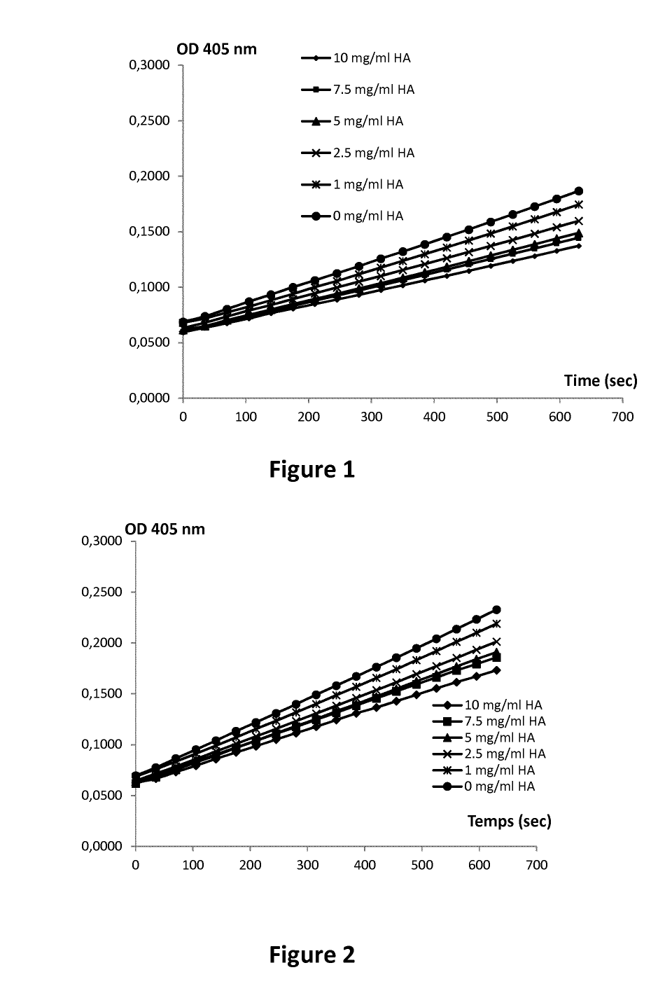

FVIIa at 2 μg/ml (FIG. 1) or 4 μg/ml (FIG. 2) was incubated in the presence of various concentrations of HA ranging from 1 to 10 mg/ml. A FVIIa substrate Pefachrome FVIIa (Cryopep) was then added at 1.56 mg/ml (2.5 mM) and the apparition of the chromogenic product was measured (the optical density was read every 30 sec at 405 nm). At the two FVIIa concentrations, the signal intensity was slightly diminished when increasing the concentration of HA. After 10 minutes of incubation, there was a 27% loss of activity for 2 μg/ml FVIIa and 26% for 4 μg/ml FVIIa at the highest concentration of HA (10 mg/ml). Thus, the presence of hyaluronic acid did not significantly affect the activity of FVIIa.

### Example 3: Effect of Hyaluronic Acid on the Clot Formation

The ability of FVIIa and fibrinogen to form a clot in the presence of HA was evaluated using thromboelastometry.

Recombinant transgenic FVIIa (Stock: 1 mg/ml, LFB) was diluted in_HEPES buffer (25 mM Hepes, 175 mM NaCl pH 7.4) in the presence of tissue factor (0.5 pM, Dade Innovin Siemens, ref. B4212-40), 2 μM phospholipids (STAGO), 3 mM CaCl₂ (Sigma), and 20 mg/ml plasma derived fibrinogen (Clottafact, LFB). Coagulation was initiated by the addition of 5% of plasma (Unicalibrator STAGO) and recorded in the Rotem apparatus (TEM). Kinetic parameters were extracted using the provided program. The composition containing plasma derived fibrinogen (at variable concentrations), FVIIa (4 μg/ml), tissue-factor (0.5 pM), phospholipids (2 μM) and calcium (3 mM) was placed in a cuvette. The reaction was initiated by adding a volume of plasma (final concentration 5%). A circular piston pin was immersed in the solution and let to rotate. As soon as the mixture begins to coagulate and when the firmness of the clot increased, the clot limited the piston pin rotation. This variation of resistance was optically detected for 60 min and transformed in typical kinetic curves (TEMogram). Curves were then automatically analyzed and several parameters of the reaction were provided, in particular the time of coagulation (CT), the time to form the clot (CFT), the maximum clot firmness (MCF) allowing the measurement of the total coagulation time (TTC=CT+CFT) and the ratio MCF/(CT+CFT) which reflects the efficiency of clotting from the mixture.

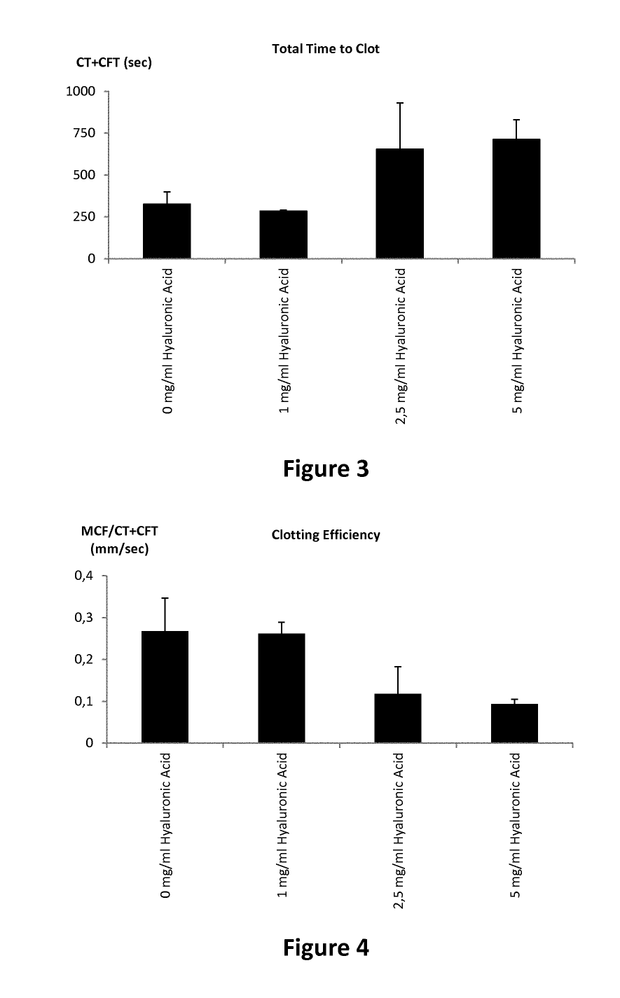

When HA was added the total time to clot (TTC) was increased in function of the concentration up to 5 mg/ml HA (FIG. 3). At low HA concentration the TTC was not affected but at the highest concentrations a 66% increase in the TTC was observed. This increase in the clotting time affects the efficiency of clotting since the ratio MCF/TTC, reflecting this efficiency, was diminished in the same proportion (FIG. 4). In conclusion, a composition comprising FVIIa and fibrinogen was not significantly affected by the presence of HA and kept the ability to clot in vitro.

1. An injectable composition comprising a filler and Factor VII, preferably Factor VIIa.

2. A composition according to claim 1, wherein said filler is hyaluronic acid.

3. A composition according to claim 1 or 2, wherein said composition further comprises fibrinogen.

4. A composition according to any one of claims 1 to 3, wherein said composition further comprises Factor XIII.

5. A composition according to any one of claims 1 to 4, wherein at least one protein selected from said Factor VII, said fibrinogen and said Factor XIII, is recombinant, preferably wherein all the proteins selected from said Factor VII, said fibrinogen and said Factor XIII, are recombinant.

6. A composition according to any one of claims 1 to 5, wherein said composition further comprises an anesthetic agent, preferably lidocaine.

7. A kit comprising i) at least one syringe and ii) a container containing a composition according to any of claims 1 to 6.

8. A composition according to any one of claims 1 to 6 for use by injection in preventing or treating skin defects, preferably folds, wrinkles, skin depressions and scars.

9. A method for preventing or treating skin defects, preferably folds, wrinkles, skin depressions and scars, by injecting in an subject in need thereof, a composition according to any one of claims 1 to 6.

10. Factor VII for use in diminishing, decreasing or avoiding skin reactions due to injection of a filler in a subject, preferably redness, ecchymosis, bruising, bleeding, erythema, oedema, necrosis, ulceration, swelling and/or inflammation, by injection to a subject simultaneously or sequentially, e.g. subsequently, to the filler.

11. Factor VII for use according to claim 10, wherein the Factor VII is combined within the injectable filler composition as defined in any of claims 1 to 6.

12. Factor VII for use according to claim 10, wherein the Factor VII is in the form of a separate injectable composition for an injection sequential to the injection of the filler.

13. The composition as defined in any of claims 1 to 6, for use in diminishing, decreasing or avoiding skin reactions due to injection in a subject, preferably redness, ecchymosis, bruising, bleeding, erythema, oedema, necrosis, ulceration, swelling and/or inflammation.

14. A method for diminishing, decreasing or avoiding skin reactions due to injection of a filler, preferably redness, ecchymosis, bruising, bleeding, erythema, oedema, necrosis, ulceration, swelling and/or inflammation by injecting the subject with Factor VII, simultaneously or sequentially, e.g. subsequently to the injection of the filler.

15. The method of claim 14, wherein the filler and Factor VII are injected in a single composition, as defined in any of claims 1 to 6.

16. A method for diminishing, decreasing or avoiding skin reactions due to injection of a filler, preferably redness, ecchymosis, bruising, bleeding, erythema, oedema, necrosis, ulceration, swelling and/or inflammation by injecting in an subject in need thereof, a composition according to any one of claims 1 to 6.

17. A kit comprising a container containing an injectable composition of a filler, and a container containing an injectable composition of FVII.
